(19) United States
(12) Patent Application Publication
(10) Pub. No.: US 2026/0240484 A1
(43) Pub. Date: Aug. 20, 2026

(54) COMPREHENSIVE EEG-BASED SUBSTANCE QUANTIFICATION

(71) Applicant: Krishna Gandhi, Greater London (GB)

(72) Inventor: Krishna Gandhi, Greater London (GB)

(21) Appl. No.: 19/162,930

(22) PCT Filed: Mar. 18, 2024
(86) PCT No.: PCT/GB2024/050736

§ 371 Date: Sep. 16, 2025

(30) Foreign Application Priority Data
Mar. 18, 2023 (GB) 2303973.8

(51) Int. Cl. A61B 5/374; A61B 5/00; A61B 5/021; A61B 5/11; A61B 5/145; A61B 5/256; A61B 5/291; A61B 5/297; A61B 5/31; A61B 5/315; A61B 5/398

(52) U.S. Cl. CPC A61B 5/374; A61B 5/021; A61B 5/1103; A61B 5/14546; A61B 5/256; A61B 5/291; A61B 5/297; A61B 5/31; A61B 5/315; A61B 5/398; A61B 5/4809; A61B 5/4812; A61B 5/6803; A61B 5/7235; A61B 5/7405; A61B 5/742; A61B 5/7475; A61B 2560/0468; A61B 2562/0219

(57) ABSTRACT

This invention presents a system using an alternative method to quantifying levels of substances from the EEG data using a non-linear equation based on the same essential features of previous disclosures. The essential features consist of predetermined frequency bands, the amalgamated ratio and a regression formula: (Formula (I)) where Y is the EEG-based substance level quantification.

### DESCRIPTION OF THE RELATED ART

This patent builds on the research share in patent GB2492852 and PCT/IB2023/050795. GB2492852 had demonstrated the ability to measure cortisol from the EEG and PCT/IB2023/050795 the ability further improve the accuracy of measuring substance levels from the EEG data along with a serviceable system that incorporates many features related to the measuring substance levels such as hormones, neurotransmitters and biomarkers from the EEG. A follow-on study carried out in April 2015 based on the study published in PCT/IB2023/050795 discloses the ability to measure multiple substance such as Testosterone, Estrogen, ACTH as well as an improving the accuracy of Cortisol from GB2492852, although Cortisol in GB2492852 acquired cortisol from salivary samples.

### FIELD OF THE INVENTION

Embodiments of the present invention relates to a system within the field of health technology and bioinformatics. More particularly, the present application relates to methods, apparatus and systems for quantifying levels of substances non-invasively from the electroencephalography EEG data.

GB2492852 considered the various biomarkers the EEG has been used for and focused on the references specific to its invention. In previous disclosures, the method and system is configured to determine the average powers of the predetermined frequency band of a substance from which an amalgamated ratio and therefore the linear regression line is derived to obtain a value for a level of said substance.

PCT/IB2023/050795 builds on the method to measure substance levels from the EEG and provides details on how to enhance the accuracy of each essential feature consisting of electrode location(s), predetermined frequency band(s), where k-cluster at 1 HZ bandwidths against substance levels are applied to determine the electrode locations and predetermined frequency bands, this impact accuracy of the amalgamated ratio where the disclosure was use AI or machine learning to speedily derived the correlated combination of the predetermined frequency bands with the substance and select one to derive the general linear equation to measure levels of said substance and disclosed factors such as gender necessary for improving the accuracy of measuring levels of substance from the EEG brain data. Since the underlying dynamics are not evident without research, how to apply quantitative methods are not obvious. In the case of PCT/IB2023/050795, auto-regression and error may seem relevant to apply in process to derive an improved method to measure levels of substance from the EEG.

PCT/IB2023/050795 incorporates gender in the analytical pipeline presented in GB2492852 which is further enhanced by taking other factors into consideration such as sleep-wake states, sleep stages and age. Both sleep-wake and gender groups demonstrate different operations which also correlate to significantly different activity between hormones and neurotransmitters which provides the basis to run the data analyses that include either gender, sleep-wake-state, handedness (left, right or ambidextrous), or any indication of hemispheric dominance to optimise the algorithm to measure or provide an indication of the level of substance required to measure.

Unlike innovation and research based on applied AI in large datasets as a blanket approach, the research a carried out at Psimonix applies the enhancement through the logical progressed insights. The insights are valuable for configuration settings to optimized AI or machine learning and reduce the time and data required to derive said goal. Psimonix research has impacted the approach and methods or scientific publication in the field of neuro technology.

The method includes the steps of obtaining and analysing data to determine at least one average power for each of a plurality of predetermined frequency bands, performing a general linear algebra operation on the at least one user characteristic based on at least three average powers to model a relationship between an actual level of said at least three levels of a substance and a predicted level of the at least three levels of substance, applying trained artificial intelligence (AI) model that is adapted to analyze collected data and modeled relationship based on known configuration to determine the at least one substance level in the body, comparing the results that are statistically significant to determine the at least one substance level in the body of the subject, and select any one significant result that is reasonable to determine the at least one substance level.

The signal generation detected from the EEG are associated with general health, performance, well-being, mental health, mental state, emotions and which can be logically extrapolated to include thoughts and all that is being experienced or perceived from within and from the information processed by the senses.

As mentioned in GB2492852, the role of cortisol and psychological assessment to biomarkers, for example and not limited to alcoholism, ADHD and dementia, and the significance of the HPA regulation. The EEG applied in neurofeedback, provides a method to reregulate from preceding mentioned ailments and many others. The configurations are vast which limits the success rate of neurofeedback effectiveness. Additionally there are many visual and auditory forms for the feedback to be relayed. A visual example of neurofeedback is displayed of a car on a track where it responds to the user's EEG data acquired by a device. The feedback is rewarded based on the training protocol configured.

The Quantitative EEG (QEEG) techniques include the computation of power and associated scalp topographic maps for given frequency bands. Such techniques have been used during the past three decades to illustrate, diagnose and investigate neuropsychological states such as depression, alcoholism, schizophrenia or cognitive functions such as attention disorders, memory and vastly used to diagnose epilepsy.

### SUMMARY OF THE INVENTION

This invention embodies an alternative method to quantify levels of substances from the EEG from a non-linear equation based on the same essential features. This alternative, if not a second incremental improved method is an outcome of a comparative analysis of the data used in disclosures of PCT/IB2023/050795, wherein substances in the present invention includes hormones, neurotransmitters, neuromodulators, biomarkers and linked ailments of substances to EEG digital markers of hormones, neurotransmitters and neuromodulators of the data originator. Further embodiments include components of the system that applies EEG derived quantified substance levels in a serviceable system. These components include the Software as a Service or platform, a neurofeedback feature and a blockchain feature. The invention comprises an EEG wearable apparatus to acquire the EEG data that can integrate with the system and the neurofeedback, blockchain component. The present invention builds on the general settings of the system of PCT/IB2023/050795 to measure levels of substances from the EEG data and derives the EEG algorithm for quantified levels of substances with mentioned and new specified methods and feature optimization.

### DETAILED DESCRIPTION

### Method:

Whilst providing a serviceable product; PsiSense to screen hormones levels and with a validation study to support the EEG to be a reliable tool to screen for hormones levels, in a few occasions results returned would exceed the limits of possible numbers. In investigating this, there were at least two reasons including but not limited to noisy EEG data or because consideration of maximum and minimum were missed using a general linear equation.

Given that at least for cortisol there is consistent research to demonstrate the associated delta and beta frequency bands powers; where power can be the total amplitude at least or power spectral density or spectral power, and therefore the predetermined frequencies of said substance, the biological maximum or minimum would not be exceeded. Reviewing the data again it is visible that what looks like linear correlation for the most part are actually in some cases, logarithmic or exponential such examples are seen in plots from previous study, see FIG. 1. In The scatterplot of the delta and beta frequency bands against cortisol levels at O1, see FIG. 1a and O2, see FIG. 1b are seemingly logarithm with respect to delta in blue and exponential with respect to beta (orange/red). Another example of this non-linear regression relationship of predetermined frequency bands can be seen for testosterone, see FIG. 1c and FIG. 1d and more evidently where all frequency bands except for alpha band (light turquoise) are demonstrating a non-linear relationship with testosterone at O1 and O2 for males and deviating from the linear trend shown on the scatter plot.

Logarithmic and exponential functions are utilized in neuroscience to characterize neural activity and signal transmission. For example, the relationship between stimulus intensity and neural response often follows an exponential or logarithmic curve, known as the neural response function. To capture the known biological curve behaviours a flexible regression can express a sigmoidal, exponential, or logarithmic relationship between exponential correlated values and a biological related substance, a generalized form of the logistic function, also known as the sigmoid function. The sigmoid function is commonly used to model S-curve relationships and can also represent exponential and logarithmic behaviours under certain parameterisations.

To combine the frequency bands that show an exponential correlation in to an amalgamated single value to exhibits a significant correlation with the concentration levels of cortisol, a weighted sum, a polynomial regression equation or a log-log model are shown to have predictive powers, that it is consider all variable and does not assume linearity. A comparative study was carried out to test the linear model for cortisol ratioK that finds inventions disclosed in GB2492852 and PCT/IB2023/050795. A polynomial interaction study was compared to ratioK to test its theoretical significance. It was found that ratiok had a better predictive power for cortisol than log-log model. However the log-log model did show predict power for cortisol and so a consideration for having a mixed approach of linear and non-linear relations.

For frequencies are exhibiting an exponential correlation with cortisol an amalgamating approach is to calculate the geometric mean of the variables. The geometric mean is a measure of central tendency that accounts for the exponential nature of the data. By taking the geometric mean of the powers of the predetermined frequency band at each concentration level, and taking the nth root (number of predetermined frequency bands) of the product to obtain the geometric mean, represents their collective influence. For cortisol the predetermined frequency bands are: delta, beta, gamma, theta, alpha, therefore taking the ⅕th root of the product of the average powers of each predetermined frequency band over at least 3 seconds, to yield the amalgamated value, x, The geometric approach would broaden the definition of the amalgamated process and the hence the derivation of the amalgamated ratio. Further, the essential feature of the linear equation would now include the following logistic regression in the embodiments of this invention:

$$Y = \frac{L}{1 + {e}^{- k ( x - x 0 )}}$$

Where: Y is the value of the substance level, L is the maximum value of Y representing the upper asymptote, k is the growth rate parameter, controlling the steepness of the curve, x0 is the x-value of the sigmoid midpoint, where the curve reaches its inflection point, e is the base of the natural logarithm (approximately equal to 2.71828).

The generalised logistic equation allows for flexibility in capturing different shapes of relationships between the amalgamated value and said substance. Parameters L, k, x0 are adjustable to represent a curve relationship that is sigmoidal, exponential, or logarithmic. An S-curve correlation may arise when the individual exponential correlations reach a plateau or exhibit diminishing returns at extreme values of any said substance. In such cases, the combined effect of multiple variables can lead to a gradual increase followed by a steeper rise and then a flattening or saturation as a substance increases or decreases, solving the initial discrepancy of returning quantified levels of substances that are out of biological bounds and progressing towards a deeper understanding of the underlying dynamics, non-invasively.

In this invention the method to quantify levels of said substance maintains the same steps and all essential features such as at least one related electrode location, at least one predetermined frequency band(s), amalgamated ratio, and a regression function. However in this invention the essential features encompasses non-linear forms, wherein predetermined frequency bands include at least one frequency band where its average power is non-linear correlated (logarithms of exponential) with increasing or decreasing concentration levels of said substance and where the amalgamated ratio is obtain using the geometric mean of the predetermined frequency bands but limited that approach, and further wherein the regression function includes a logistic regression.

### System

The inventions of present system invention builds on that described in PCT/IB2023/050795.

The embodiments of the invention include a Software As A Service (SAAS) for a serviceable method for EEG-Based Substance Level Quantification includes component such as Neurofeedback system to regulate the substance levels in the body non-invasively and a blockchain component for providing user an option to share data and to provide service certification to ensure industry standards. Additional feature where information and links are made available to the user of ailments linked to EEG-based substance levels quantification.

### Neurofeedback

The system component described herein is a neurofeedback system designed for monitoring and regulating brain activity associated with regulated substances. It consists of an EEG device equipped with active electrodes to capture EEG signals from the user's scalp. These signals are then analyzed by a signal processing module, which extracts features related to brain activity linked with regulated substances. Real-time feedback is provided to the user through a feedback interface, which may include a scalp topography displaying brain activity distribution in 2D or 3D, audio cues conveying information about brain activity, or a visual display of numerical EEG parameters associated with regulated substances. The scalp topography offers real-time visualization of brain activity patterns related to the presence or concentration of regulated substances, enabling users to track changes over time. The feedback interface also includes a training mode where users can undergo neurofeedback training sessions to learn to modulate their brain activity in response to feedback. Additionally, the feedback integration module dynamically adjusts the feedback based on real-time changes in EEG signals, enhancing the efficacy of the neurofeedback training process. Optionally, a blood pressure monitoring device can be integrated into the system to obtain additional physiological data. A feedback integration module incorporates blood pressure measurements into the real-time feedback, optimizing the effectiveness of the neurofeedback training.

The EEG is capture at real time and quantified iteratively over a systems configured windowed average to calculate the level of the substance being trained where the display of levels as feedback are presented on screen along with an option to view a 3D scalp topology of brain activity for the timed session. Other forms of feedback can include audio.

Further embodiments include selecting the length of training session which to self assess and monitor progress. The training performance can be presented as a graph or report which can be downloaded or shared by the user.

### Device:

The EEG wearable device will have at least two electrodes on the original frame and a swivel bases at the ears. The wearable EEG monitoring module communicates preferably with the personal communication device (for e.g., smartphone).

The personal communication device according to the invention is Internet enabled which means that the personal communication device may access the Internet via a wireless Internet connection (e.g. WLAN such as 802.11a, b or g), or a cellular data connection (e.g., WCDMA or LTE, air gap). Advantageously, the personal communication device has the ability to download and launch application software from an app store via the Internet or use straight on the web browser with SAAS from FIG. 2. The term “app” is short for application software which is a set of one or more programs designed to carry out operations for a specific application.

In one embodiment, extenders include pleat style expansion and compression to facilitate flexible adjustment to fit any head side and shape. The bellows can be compressed and extended for the required electrode location position along the axis of the connection at the swivel, attached left and right of the original frame.

In one embodiment, multiple add-on electrodes can be added to the extender.

In one embodiment, multiple extenders can be added to the swivel base. The Swivel base allows the extender to move across the head.

An option to add sensor to the EEG wearable device include more EEG sensor/electrodes, electrooculography EOG electrode, magnetometer and inertial sensor unit which can transmit data for any required embodiments of this invention.

In another embodiment of the present invention, the system comprises steps of mounting at least one wearable device on a user's head, or contact with user skin to measure said at least one user characteristic, measuring EEG data with said at least one wearable device, and wherein said EEG data is collected from at least two electrode positions and receiving EEG data from said at least one wearable device via said communication network, and measuring EOG data with said at least one wearable device, and wherein said EOG data is collected from at least two electrode positions and receiving EOG data from said at least one wearable device via the communication network.

In one embodiment, the data is selected from the EEG data and the EOG data.

The wearable is registered with the neurofeedback system to be able transmit data and real-time to and EEG-based substance level quantified levels at real-time.

The GUI feature for setting configurations and accessing raw data with the option to link to the Blockchain feature.

Additionally, disclosed are wearable devices equipped with add-on electrodes for signal measurement, and a configuration module for optimizing signal recording by adjusting frequency bands, markers, references, impedance, and noise filters. The wearable device facilitates sharing of measured signals with third-party devices or applications via a graphical user interface.

### Blockchain

The blockchain employed can be configured as a smart contract, serving as an account-holding object. The smart contract is designed to certify approved EEG algorithms per substance, ensuring data integrity and security.

Moreover, in yet another embodiment, the blockchain network utilizes a decentralized application design, employing a storage and distributing file sharing platform. This design enhances the scalability and resilience of the blockchain network while facilitating secure data storage and distribution.

### FIGURES

FIG. 1: Visible exponential and logarithm relationship between frequency bands and substance levels

FIG. 1C: plot for females at O1 between frequency band power (y) and cortisol (x)

FIG. 1C: plot for females at O2 between frequency band power (y) and cortisol (x)

FIG. 1A: plot for males at O1 between frequency band power (y) and testosterone (x)

FIG. 1B: plot for males at O2 between frequency band power (y) and testosterone (x)

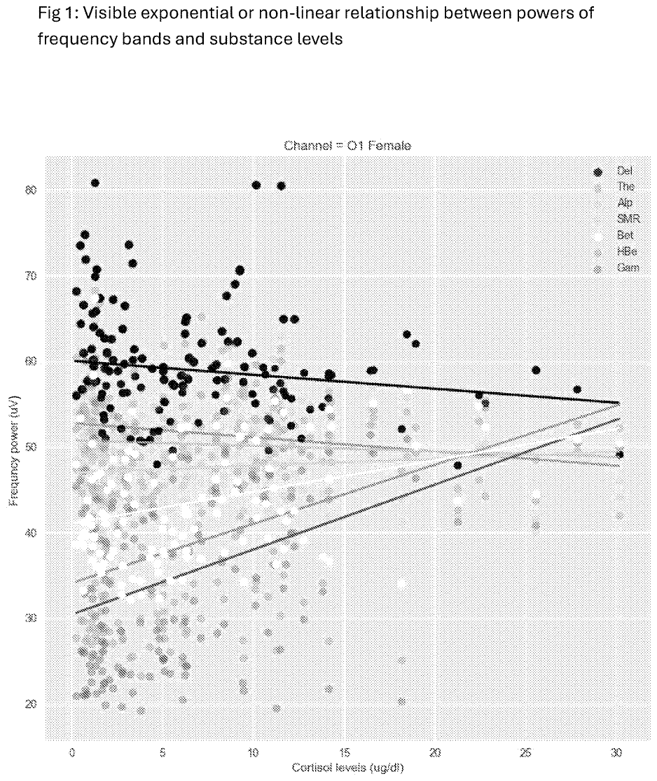

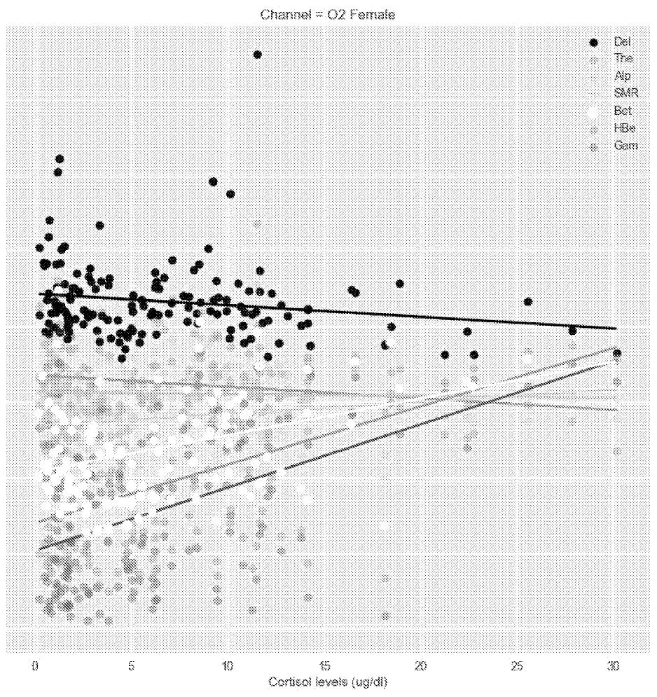

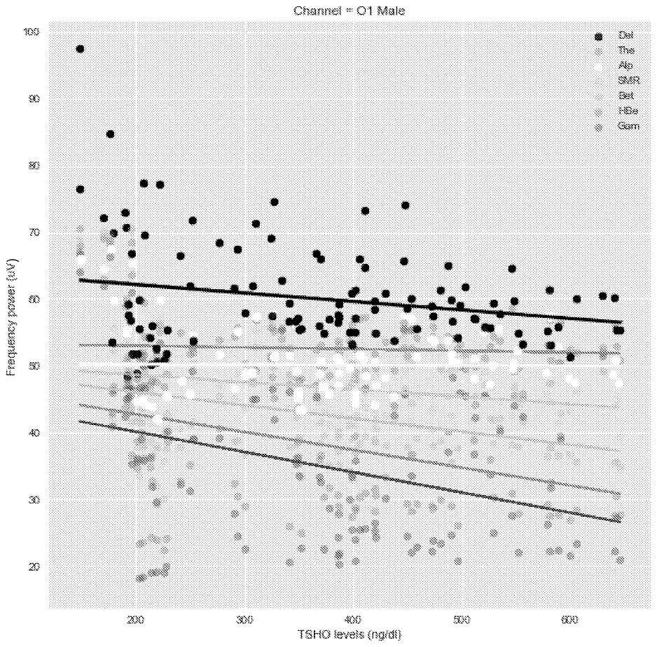

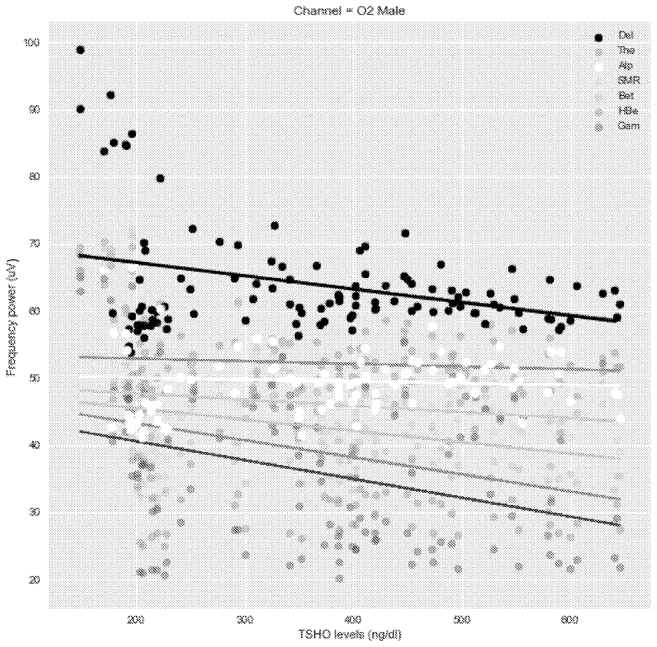

1. A comprehensive EEG-based substance level quantification system, method, comprising:
a. Method Aspect:
i. obtain EEG signal data
ii. analyzing EEG data to obtain the average power(s) for each of its said predetermined frequency band(s);
iii. calculating a value from the amalgamated ratio determined for said substance; and
iv. obtaining a quantified level of said substance from S-curve equation:
$$Y = \frac{L}{1 + {e}^{- k ( x - x 0 )}}$$
v. Optionally, adapting the method to alternative quantification methods,
wherein Y is said one substance level selected from hormones, neurotransmitters, neuromodulators, analyte and/or biomarkers.
b. System Aspect:
i. An EEG data acquisition module configured to receive or obtain EEG data;
ii. analyse said EEG data and obtain the average power(s) for the predetermined frequency band(s);
iii. calculate a value from the amalgamated ratio determined for said substance
iv. and obtain a quantified level of said substance from equation of
$$Y = \frac{L}{1 + {e}^{- k ( x - x 0 )}}$$
wherein Y is said one substance level selected from hormones, neurotransmitters, neuromodulators, analyte and/or biomarkers,
wherein Y is said one substance level selected from hormones, neurotransmitters, neuromodulators, analyte and/or biomarkers and wherein the claim encompasses both existing and earlier quantification approaches, regardless of the specific implementation

2. A method as claimed in claim 1, wherein said method further comprises the steps of:
a. obtaining the birth gender to determine the predetermined frequency bands for the substance to quantify
b. analyzing EEG data to obtain the average power for each of said predetermined frequency band(s);
c. calculating a value from the amalgamated ratio determined for said substance; and
d. obtaining a quantified level of said substance from S-curve equation:
$$Y = \frac{L}{1 + {e}^{- k ( x - x 0 )}}$$
wherein Y is said substance level is selected from hormones, neurotransmitters, neuromodulators, analyte and/or biomarkers.

3. The method as claimed in claims 1 and 2 wherein said method further comprises the steps of:
a. collecting at least one data parameter
b. analyzing EEG data to obtain the average power(s) for each of its said predetermined frequency band(s);
c. calculating a value from the amalgamated ratio determined for said substance; and
d. obtaining a quantified level of said substance from S-curve equation:
$$Y = \frac{L}{1 + {e}^{- k ( x - x 0 )}}$$
wherein said at least one data parameter is selected from handedness, age, sleep-wake state and sleep stage.

4. The method as claimed in claim 3, wherein obtaining:
a. EOG data along with the EEG data to determine at least one data parameter from sleep-wake state and sleep stage.
b. handedness information from stored data or determined through the EEG data
c. gender information from stored data or determined through at least one of EEG and EOG data
d. age from stored data or determined from the EEG

5. A system as in claim 1 further comprising:
a. receive EEG data;
b. analyse said EEG data and obtain the average power(s) for the predetermined frequency band(s);
c. calculate a value from the amalgamated ratio determined for said substance
d. and obtain a quantified level of said substance from equation of Y=bX+C
wherein Y is said one substance level selected from hormones, neurotransmitters, neuromodulators, analyte and/or biomarkers.

6. In a method or system according to any one of the preceding claims, wherein essential features of said methods are derived by AI-integrated configuration or machine learning processes from leading research insights embodied in previous linked patents and optimized to provide enhanced accuracy outcomes that quantify levels of substances from the EEG data and wherein said method is fully automated.

7. The system as claimed in claim 5 comprising of a software as a service, described in PCT/IB2023/050795 which describes the features and technical requirements for a comprehensive EEG substance quantification system such as:
a. a sub-module configured to retrieve user data from an EEG database, an EOG database and said user data is computed to determine at least one EEG algorithm per substance
b. a historical sub-module configured to present said user data in a graph format, and/or a report to view historical data and retrieve previous history
c. a registration module for an individual user or business clients
d. a module for API based connections to a third party service providers to access the ability to measure substance levels from EEG data.
e. determination sub-module configured to determine whether user data has been derived for said at least one substance level;
f. an analysis sub-module configured to retrieve user data from an EEG database, an EOG database and said user data is computed to determine at least one EEG algorithm per substance; and
g. a receiver module from a device required for quantifying substance levels from the at least EEG data
h. a method to send report or EEG data via email.
i. a method to send report or EEG data via blockchain
j. a prepaid credit system enabling users to pay for the service
k. displays of reference range information and status indicator is displayed with the levels of substances and additionally links to information linked to the substance levels and possible ailments.
l. attachment of certification for algorithm used for substance measure to ensure industry standards.
m. an opt in or out feature for users can be given the option to consent to share their health data with healthcare professionals, enabling professionals to review the information and provide appropriate guidance.

8. A system of claim 5, wherein a neurofeedback system comprising:
a. an EEG device configured to obtain electroencephalogram (EEG) signals from a user's scalp using at least one active electrode;
b. computing the average frequency power for each band is obtained based on measurements. Predetermined frequency bands are selected, considering gender, age, sleep-wake state, and handedness.
c. a signal processing module configured to analyze the EEG signals and extract features related to brain activity associated with regulated substances;
d. a feedback interface providing real-time feedback to the user based on the analyzed EEG signals, the feedback comprising at least one of:
i. a scalp topography displaying the spatial distribution of brain activity in 3D;
ii. audio feedback conveying information about brain activity through sound cues;
iii. visual display of numerical values representing EEG parameters associated with regulated substances; and providing real-time measure of EEG quantified level of substances in the users body.
e. The neurofeedback system of claim 1, wherein the scalp topography provides a real-time visualization of brain activity patterns associated with the presence or concentration of regulated substances, allowing users to monitor changes in brain activity over time.
f. The neurofeedback system of claim 1, wherein the feedback interface further comprises:
g. a training mode wherein users can undergo neurofeedback training sessions to learn to modulate their brain activity patterns in response to feedback related to regulated substances
h. The neurofeedback system of claim 1, wherein the feedback integration module is configured to dynamically adjust the feedback provided to the user based on real-time changes in EEG signals thereby optimizing the effectiveness of the neurofeedback training.
i. an optional blood pressure monitoring device configured to obtain blood pressure measurements from the user;
i. a feedback integration module configured to incorporate blood pressure measurements into the real-time feedback provided to the user.

9. A system of claim 5, where there is a blockchain-based application comprising:
a. a user interface enabling registered users to engage with virtual goods or services provided by service providers, the user interface facilitating transactions and interactions between users and service providers;
b. a certification module operated by service providers to certify the quality, authenticity, or compliance of their virtual goods or services, the certification information recorded on a distributed ledger;
c. a data sharing feature allowing users to opt in or out of sharing their transactional data, including certification information, with third parties, the data sharing preferences recorded on the blockchain ledger;
d. a rewards mechanism wherein users who opt in to share their transactional data, including certification information, are eligible to receive benefits, incentives, or rewards from third parties accessing the data.

10. An apparatus of claim 1, where a wearable EEG device comprising:
a. a headphone-like structure designed for comfortable wear on a user's head,
b. a swivel mechanism located at the base of the device, allowing for rotation and adjustment
c. extendable ports integrated into the swivel mechanism of either side of the head, facilitating the attachment of additional electrodes, enables customizable positioning of the electrodes for optimal contact and signal acquisition.
d. EEG (Electroencephalography) sensors embedded within the headphone structure for capturing brain activity,
e. EOG (Electrooculography) sensors integrated alongside the EEG sensors for simultaneous monitoring of eye movements,
f. With the ability to add multi sensors such as inertial sensor unit or a magnetometer.
g. The wearable EEG device of claim 1, wherein the extendable ports are configured to attached to add-on electrodes, enabling the attachment of supplementary electrodes for customized electrode configurations.
h. The wearable EEG device of any preceding claim, wherein the EEG sensors and EOG sensors are connected to a data acquisition unit housed within the device, facilitating real-time data recording and transmission.
i. The wearable EEG device of any preceding claim, further comprising a wireless connectivity module for seamless data transfer to external devices such as computers or smartphones
j. to acquire and send data to said system.
k. an interactive interface linking device to configure and view settings, including and not limited to filter, reference, montage settings and impedances checks.
l. An apparatus that transmit data at real time to the neurofeedback system
